(19) United States
(12) Patent Application Publication
(10) Pub. No.: US 2014/0252177 A1
(43) Pub. Date: Sep. 11, 2014

(54) MULTIPLE INTRAVENOUS LINE ORGANIZER AND METHOD FOR USING THEREOF

(71) Applicant: Elmer V. Vera, American Canyon, CA (US)

(72) Inventor: Elmer V. Vera, American Canyon, CA (US)

(21) Appl. No.: 14/193,486

(22) Filed: Feb. 28, 2014

Related U.S. Application Data
(60) Provisional application No. 61/851,453, filed on Mar. 8, 2013.

(51) Int. Cl. A61M 5/14

(52) U.S. Cl. CPC A61M 5/1418
USPC 248/68.1

(57) ABSTRACT

A multiple intravenous line organizer for managing multiple intravenous lines which provide a mechanism for labeling each line, prevent the lines from tangling, and place the injection port in an accessible position thereby preventing errors associated with simultaneous multiple intravenous drug infusion.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

1. Field of the Invention

The invention described herein generally relates to intravenous line organizers and more specifically it relates to a multiple intravenous line organizer for managing multiple intravenous lines which provide a mechanism for labeling and identifying each lines, prevent drug incompatibility reactions (when incompatible drugs are administered in the same line), prevent accidental bolus of potent drugs (when wrong IV line is used to administer emergency medication), prevent the lines from tangling and place the injection port in an accessible position thereby preventing medication errors associated with simultaneous multiple intravenous drug infusion.

2. Description of the Related Art

In a hospital setting, and specifically in a critical care setting, patients are often treated and medicated by introducing therapeutic agents, drugs, medicines, nutrients, and various other liquids directly into the blood stream through systems commonly known as intravenous (IV) administration systems. In the critical care setting, often a central venous catheter is employed to deliver a wide range of drug dosages. An IV administration system consists of a length of transparent IV tubing; one end is adapted to be inserted into a supply reservoir containing a prescribed medication or other prescribed liquid. The reservoir may be in the form of a bag or a bottle, and is generally suspended from a pole so as to be above the level of the patient. The flow of fluid is facilitated by an IV infusion pump, a sophisticated therapy-specific device used primarily for fluids, nutrition and cardiovascular drugs. One characteristic of an IV infusion pump is its capability for a wide range of acceptable programming parameters. They can deliver a drop or two over one hour, or they can be programmed to deliver one liter or more in an hour. The other end of the IV tubing is adapted to be inserted into an intravenous catheter whose catheter tip dwells into major blood vessels of the patient such as superior vena cava. This intravenous catheter is commonly called a central venous catheter.

It is not uncommon for a plurality of intravenous lines, each connected to a different source of intravenous fluid, to be used simultaneously to deliver several medicaments at once to a single patient. As a result, the simultaneous use of multiple intravenous fluid lines can create a problem with the identification of a particular medicament source with a particular medicament output. Quick identification of a medicinal fluid source is often required in emergency situations. For example, when a patient hooked up to multiple intravenous lines is in need of emergency intravenous administration of a medication not currently being provided through the intravenous lines, it is necessary to immediately provide that medication. This involves identifying the emergency medication port. Failure to take this precaution can lead to deleterious effect when administered to a line which is supposed to deliver a few drops of medication hourly.

This problem is aggravated by the tendency of each of the intravenous lines to coil and consequently tangle with other lines. The entanglement of numerous IV administration systems is common in the hospital room while the patient is recovering from a surgical operation. This entanglement can be by the patient moving around in either a conscious or unconscious state. This problem is aggravated by the tendency of each of the IV tubes to coil and consequently tangle with other IV tubes. Thus there is a need for a simple but effective means to both organize and protect intravenous lines and IV tubes to ensure an organized yet functional arrangement.

### SUMMARY OF THE INVENTION

The present invention provides a method and system for organizing an intravenous line comprising an IV line organizer and accessory parts. An object is to provide a multiple intravenous line organizer for managing multiple intravenous lines which provide a mechanism for labeling each line, prevent the lines from tangling and place the injection port in an accessible position thereby preventing errors associated with simultaneous multiple intravenous drug infusion. Another object is to provide a multiple intravenous line organizer that organizes and separates intravenous lines. Another object is to provide a multiple intravenous line organizer that provides a mechanism for labeling intravenous lines. Another object is to provide a multiple intravenous line organizer that prevents tangling of lines. Another object is to provide a multiple intravenous line organizer that provides quick access to an emergency injection port.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1 illustrates an upper perspective view of a multiple IV line organizer according to an embodiment of the present invention;

FIG. 2 illustrates a top view of an IV line being pressed through the slits into bilateral holes according to an embodiment of the present invention;

FIG. 3 illustrates a top view of a plurality of IV lines according to an embodiment of the present invention; and

FIG. 4 illustrates a diagram of operation of a multiple IV line organizer in relation to a medication bag, infusion pump, patient, and IV line according to an embodiment of the present invention.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the present invention. Likewise, a reasonably broad scope for claimed or covered subject matter is intended. Among other things, for example, subject matter may be embodied as methods, devices, components, or systems. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

Turning now descriptively to the drawings, in which similar reference characters denote similar elements throughout the several views, the figures illustrate an IV line organizer and accessory parts according to embodiments of the present invention.

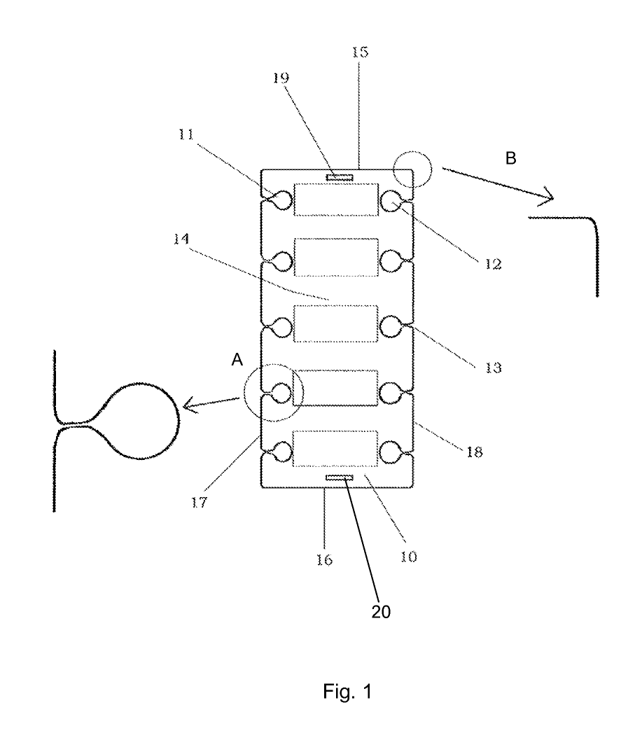

FIG. 1 presents an intravenous line organizer 10 for holding, separating, and organizing a plurality of intravenous lines. The intravenous line organizer 10 may be constructed from a transparent thin, flat sheet flexible material (such as polycarbonate). In other embodiments, the intravenous line organizer may be constructed from paper, cardboard, or any other material suitable that would be apparent to one of ordinary skill in the art. The material is preferably made of transparent material for ease of visualization of IV lines but a transparent or opaque material can also be used. The intravenous line organizer 10 includes a plurality of openings 11, 12 along the edges of the organizer for receiving IV lines and slits which extend through the edge of the organizer to the openings 11, 12 for enabling the IV lines to be inserted through the slits (shown in greater detail in view A). The slits may be constructed in such a way that allows an IV line 22 to be easily pushed through to the openings from the slits. The size of the openings are sized to accommodate IV lines of various shapes and/or sizes and large enough to prevent deformation or crushing of the IV lines. According to one embodiment, the intravenous line organizer may have smooth rounded corners as illustrated in view B.

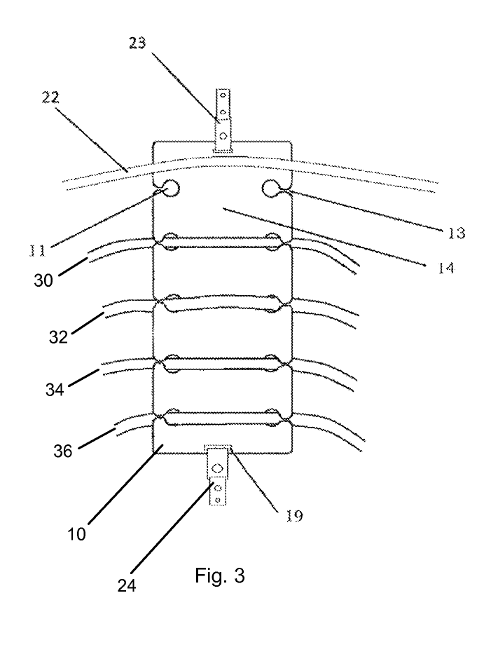

The plurality of openings on both the left and the right lateral edges 17, 18 is adapted to receive IV lines and the slits 13 which extend to the edges of the organizer into the openings enable the IV lines to be inserted through the slits and into the openings 11, 12. Clips or fasteners on both ends 15, 16 can be attached through clip holes 19, 20 to the patients gown, a rolled towel, bed sheet or even taped to the side rails as illustrated in FIG. 3. Other suitable means of attaching the IV organizer such as hook and loop (Velcro) fasteners or adhesive backing can also be used. The slits leading into the opening openings, as well as the corners of the IV organizer can be easily disinfected and is reusable.

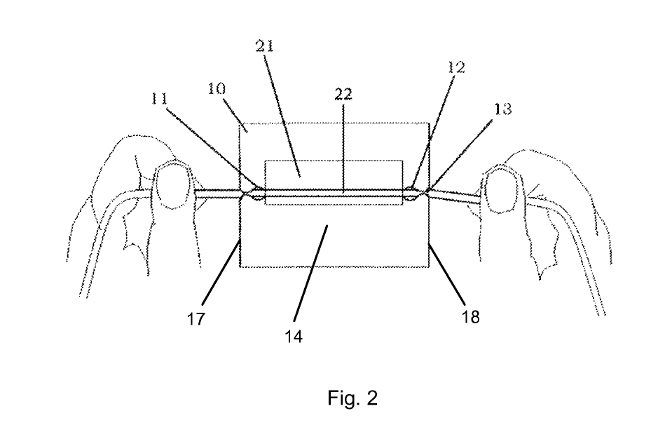

The intravenous line organizer 10 allows nurses and doctors to identify, verify and separate multiple IV lines. As illustrated in FIG. 2, an IV line 22 can be pressed through slit 13 located on both the lateral ends 17, 18 of the organizer 10 into the openings 11, 12 so that the IV line 22 is held in parallel relationship. The IV line 22 extends on top of the central portion 14 of the organizer. An adhesive label 21 (which can be writable, erasable, and/or permanent) can be placed between the openings on a top or anterior facing side of the organizer to identify the medication or fluid supplied by a given IV line. One alternative to a label is to print, using white ink on the opposite or posterior side of the clear plastic, employing silk screen process so that the top side would have a smooth and even writable surface which is advantageous since no bacteria is harbored between the adhesive of a label and plastic surface.

FIG. 3 presents a top view of a plurality of IV lines according to an embodiment of the present invention. The IV organizer can be designed to hold any number of IV lines. IV lines 30, 32, 34, and 36 may be simultaneously held and organized on intravenous line organizer 10. The intravenous line organizer is configurable to manage multiple intravenous lines which provide a mechanism for labeling each line and prevents the lines from tangling. In other embodiments, multiple organizers can be used.

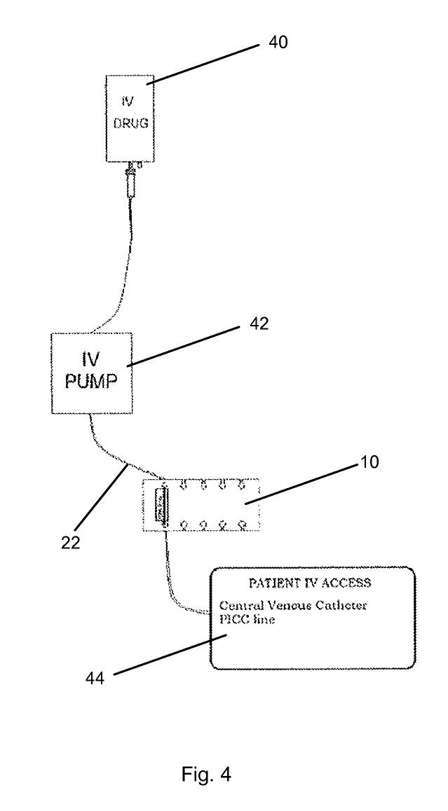

FIG. 4 presents a diagram of operation of a multiple IV line organizer in relation to a medication bag, infusion pump, patient, and IV line according to an embodiment of the present invention. IV drug 40 may include a bag with a given fluid to be provided to a patient. IV pump 42 may be located downstream from the IV drug 40 and includes an infusion pump that is operable to administer fluids in a controlled manner in a steady and/or periodic basis where volumes may vary by the time of day. IV line 22 may be connected to IV pump 42 to deliver the fluid to the patent by the patient IV access 44 (injection port). In the illustrated example, patient IV access 44 comprises a central venous catheter PICC (peripherally inserted central catheter) line. IV line 22 may be fitted onto IV line organizer 10 that provides a mechanism for labeling intravenous lines and thereby preventing errors associated with simultaneous multiple intravenous drug infusion. IV line organizer 10 may also provide identification and quick access to an emergency injection port.

FIGS. 1 through 4 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present invention have been described above, it should be understood that they have been presented by way of example, and not limitation. It would be apparent to one skilled in the relevant art(s) that various changes in form and detail could be made therein without departing from the spirit and scope of the invention. Thus, the present invention should not be limited by any of the above-described exemplary embodiments, but should be defined only in accordance with the following claims and their equivalents.

What is claimed is:

1. An intravenous line organizer for separating multiple intravenous lines and providing identification of the intravenous lines, the intravenous line organizer comprising:
a transparent, thin, flat sheet of flexible material comprising a substantially flat, planar, anterior surface portion, and an edge;
a plurality of openings formed along the edge;
a plurality of slits extending from each opening through the edge, the organizer being made of a material having sufficient flexibility to enable lengths of intravenous lines to be pressed through respective slits and into respective openings, whereby a plurality of separate intravenous lines can be engaged in respective openings to hold the intravenous lines in separated and organized relationship; and
a plurality of labels situated between the openings to provide identification of the respective intravenous lines.

2. The intravenous line organizer holder of claim 1, wherein the transparent, thin, flat sheet of flexible material is made from polycarbonate.

3. The intravenous line organizer of claim 1, wherein the openings have a size to accommodate the intravenous lines.

4. The intravenous line organizer of claim 1 comprising smooth rounded corners.

5. The intravenous line organizer of claim 1 further comprising clip holes on ends of the intravenous line organizer that are operable to receive means for attachment.
